(19) United States
(12) Patent Application Publication
(10) Pub. No.: US 2026/0041313 A1
(43) Pub. Date: Feb. 12, 2026

(54) MEDICAL DEVICES AND SYSTEMS FOR CUTTING TISSUE

(71) Applicant: Boston Scientific Scimed, Inc., Maple Grove, MN (US)

(72) Inventors: Kirsten VIERING, Newton, MA (US); Louis J. BARBATO, Franklin, MA (US)

(73) Assignee: Boston Scientific Scimed, Inc., Maple Grove, MN (US)

(21) Appl. No.: 19/284,977

(22) Filed: Jul. 30, 2025

Related U.S. Application Data
(60) Provisional application No. 63/680,191, filed on Aug. 7, 2024.

(51) Int. Cl. A61B 1/012; A61B 1/00; A61B 1/005; A61B 1/05; A61B 1/06; A61B 18/00; A61B 18/14

(52) U.S. Cl. CPC A61B 1/0125; A61B 1/0008; A61B 1/00124; A61B 1/00137; A61B 1/0052; A61B 1/05; A61B 1/0676; A61B 18/1492; A61B 2018/00083; A61B 2018/00107; A61B 2018/00178; A61B 2018/00601; A61B 2018/0091; A61B 2018/00982; A61B 2018/1407

(57) ABSTRACT

A medical device may include a handle and an insertion portion extending from the handle. The insertion portion may include a shaft and a cap. The shaft may have a proximal end and a distal end. A distal portion of the shaft may include an articulation portion. The articulation portion may be configured to bend the distal portion of the shaft in at least two directions. The cap may be fixed to the distal end of the shaft. The cap may include an imaging device, a lighting element, and an electrode. The electrode may be configured to cut a material using a high-frequency electrical current.

### CROSS-REFERENCE TO RELATED APPLICATIONS

This application claims the benefit of priority to U.S. Provisional Application No. 63/680,191, filed on Aug. 7, 2024, which is incorporated by reference herein in its entirety.

### TECHNICAL FIELD

This disclosure relates generally to medical devices and systems and, more particularly, to medical devices and systems for cutting tissue.

### BACKGROUND

Medical devices, including cutting wires, sphincterotomes, hot knives, etc., may be used to cut tissue, for example, endoscopically. These medical devices are often used in conjunction with a delivery device. Manipulating an end of such a medical device, oftentimes in addition to manipulating a position of the delivery device, can be difficult and can result in damage to nearby tissue. There is a need, therefore, for improved medical devices or systems that are configured for cutting tissue endoscopically.

### SUMMARY OF THE DISCLOSURE

Each of the aspects disclosed herein may include one or more of the features described in connection with any of the other disclosed aspects. Aspects of the disclosure relate to, among other things, medical devices or systems configured to cut tissue, for example, endoscopically.

According to an example, a medical device may include a handle and an insertion portion extending from the handle. The insertion portion may include a shaft and a cap. The shaft may have a proximal end and a distal end. A distal portion of the shaft may include an articulation portion. The articulation portion may be configured to bend the distal portion of the shaft in at least two directions. The cap may be fixed to the distal end of the shaft. The cap may include an imaging device, a lighting element, and an electrode. The electrode may be configured to cut a material using a high-frequency electrical current.

Any of the devices disclosed herein may include any of the following features, alone or in any combination. The imaging device, the lighting element, and the electrode may be disposed on a distalmost surface of the cap. The electrode may be ring-shaped. The electrode may at least partially encompass both the imaging device and the lighting element. The electrode may extend distally from a distalmost surface of the cap. A diameter of the electrode may be less than a diameter of the cap.

The electrode may extend around at least a portion of a radially outward-facing surface of the cap. A radially outward-facing surface of the electrode may be radially aligned with a radially outward-facing surface of the cap.

A distal face of the electrode may be proximal of a distalmost surface of the cap. A diameter of the electrode may be greater than a diameter of the cap. At least a portion of the electrode may be coated with an insulating material.

The medical device may be a first medical device. The insertion portion may be configured for insertion through a working channel of a second medical device.

The handle may include an electrical port electrically coupled to the electrode. The electrical port may be electrically coupled to the electrode via a steering element extending distally from the handle. The electrode may be disposed longitudinally along a radially-outward facing surface of the cap.

According to another example, a distal end portion of a medical device may include an articulation portion and a cap portion fixed to a distal end of the articulation portion. The cap may include an imaging device, a lighting element, and an electrode. The electrode may be configured to cut tissue using a high-frequency electrical current. The electrode may be disposed on a radially outward-facing surface of the cap portion.

Any of the devices disclosed herein may include any of the following features, alone or in any combination. The imaging device and the lighting element may be disposed on a distalmost surface of the cap portion. A diameter of the electrode may be greater than a diameter of the cap portion.

According to another example, a medical system may include a first medical device and a second medical device. The first medical device may include a first handle and a first shaft extending from the first handle and having a working channel. The second medical device may include a second handle and a second shaft extending from the second handle. The second shaft may be configured for insertion through the working channel of the first medical device. A distal end portion of the second shaft may include a cap. The cap may include: (i) an imaging device; (ii) a lighting element; and (iii) an electrode. The electrode may be configured to cut a material using high-frequency electrical currents.

Any of the devices disclosed herein may include the following feature. The electrode may be positioned proximal of the imaging device and the lighting element.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A and 1B depict proximal and distal portions, respectively, of an exemplary first medical device, according to aspects of this disclosure.

FIG. 2 depicts a medical system, including the first medical device of FIGS. 1A and 1B and a second medical device, according to aspects of this disclosure.

FIG. 3A illustrates a perspective view of a distal end portion of the first medical device of FIGS. 1A and 1B, according to aspects of this disclosure.

FIG. 3B illustrates a side view of the distal end portion of FIG. 3A, according to aspects of this disclosure.

FIG. 3C-3E illustrate side views of various alternative configurations of a distal end portion of the first medical device, according to aspects of this disclosure.

FIG. 4 illustrates an internal view of a handle of the first medical device of FIGS. 1A and 1B, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is or is not circular. The term “distal” refers to a direction away from an operator, and the term “proximal” refers to a direction toward an operator. Proximal and distal directions are labeled with arrows marked “P” and “D”, respectively, throughout various figures. The term “exemplary” is used in the sense of “example,” rather than “ideal. ” The term “approximately,” or like terms (e.g., “substantially”), includes values +/−10% of a stated value. Unless otherwise stated, ranges disclosed herein include the end points of the ranges.

The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element.

A first medical device, such as a cholangioscope (or other type of daughter scope) or an accessory instrument, may be inserted into a working channel of a second medical device, such as a duodenoscope (or other type of mother scope). A distal portion of the shaft of the first medical device may extend through the working channel of the second medical device. A proximal portion of the shaft of the first medical device may be proximal (or otherwise outside) of a working channel port of the second medical device. Thus, the proximal portion of the shaft may be outside of the working channel. The proximal portion of the shaft may be external to a body lumen of a subject, whereas the distal portion of the shaft may be inserted into the body lumen of the subject.

In conventional procedures, accessory devices may be inserted through a working channel of the first medical device and/or the working channel of the second medical device to cut tissue, for example, endoscopically. In some procedures, cutting the tissue may provide ease of access into parts of the anatomy that may otherwise have been difficult or impossible to reach. The accessory devices configured to cut the tissue may include a sphincterotome, a hot knife, or a similar device that utilizes an electrode or cutting device to cut tissue. Oftentimes, these devices lack maneuverability, or steering capabilities, thus presenting challenges for the user. Such challenges may result in increased procedural times, increased costs, increased risks to the subject, damage to surrounding tissue, etc. Additionally or alternatively, maneuvering and/or maintaining a position of multiple devices simultaneously (e.g., the accessory device, the daughter scope, and/or the mother scope) may present challenges for a user.

The disclosed aspects include a medical device having a cutting device incorporated into a distal end of the medical device. The cutting devices discussed herein may include monopolar and/or bipolar high-frequency cutting devices. The medical device may have steering capabilities in one or more directions, thus providing for a steerable cutting device. In aspects, the medical device may also have illumination and/or imaging capabilities. Incorporating a cutting device into a distal end of the medical device may help improve performance of the cutting device by enabling the cutting device to be steerable. Furthermore, incorporating a cutting device into a distal end of the medical device may help decrease the number of devices that may need to be maneuvered simultaneously during a procedure. In these aspects, one or more of the challenges discussed above may be alleviated. The disclosed embodiments may also include a handle of a medical device that is configured for electrical connection to a high-frequency generator, an electrosurgical unit, or similar device.

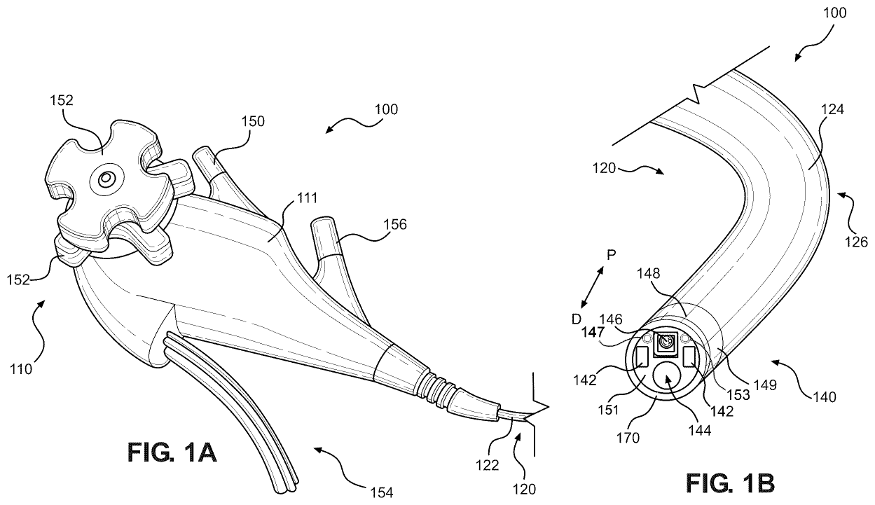

FIGS. 1A and 1B depict proximal and distal portions, respectively, of a first medical device 100. In some examples, first medical device 100 may be a cholangioscope. However, although cholangioscopes are referenced herein, it will be appreciated that first medical device 100 may be another type of scope, an endoscope, a ureteroscope, a duodenoscope, a gastroscope, an endoscopic ultrasonography (“EUS”) scope, a colonoscope, a bronchoscope, a laparoscope, an arthroscope, a cystoscope, an aspiration scope, etc.

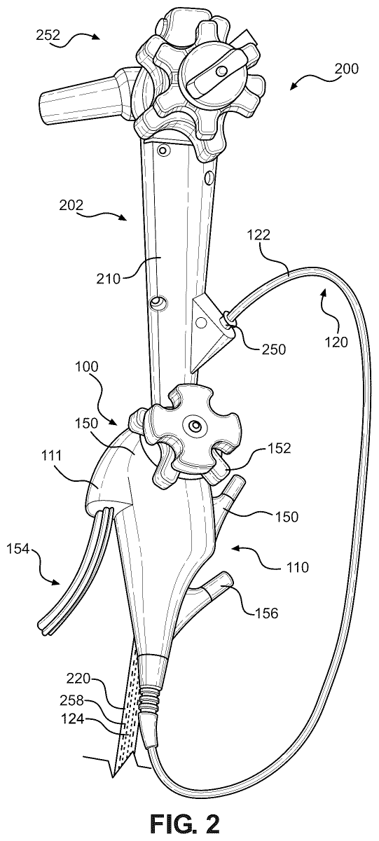

First medical device 100 may include a handle 110 and a shaft 120. Shaft 120 may have a distal end portion 140 coupled to a distal end of shaft 120. Distal end portion 140 may include a cap portion 148. Shaft 120 may include a proximal portion 122 (FIG. 1A) and a distal portion 124 (FIG. 1B). Distal portion 124 of shaft 120 and distal end portion 140 may be configured for insertion into a body lumen of a subject. For example, distal portion 124 of shaft 120 and distal end portion 140 may be inserted into a working channel of a second medical device (e.g., second medical device 202, shown in FIG. 2 and described below). As described in further detail below, proximal portion 122 of shaft 120 may remain outside of/external to the body lumen of the subject and the working channel of the second medical device. Distal portion 124 may include a steerable section 126, which may include an articulation joint or similar structure (e.g., a multilumen extruded shaft or other bendable shaft) that is configured to deflect or otherwise articulate, or bend, in order to steer steerable section 126 and distal end portion 140. For example, steerable section 126 may be configured to articulate, or bend, in one or more directions (e.g., one or more of up, down, left, or right directions).

Distal end portion 140 may include one or more lighting elements 142 (e.g., two lighting elements 142), one or more imaging devices 146, and a distal opening 144 of a working channel of first medical device 100. In some aspects, distal end portion 140 may further include one or more fluid delivery openings 147, or lumens. Fluid delivery openings 147 may be configured to deliver a liquid (e.g., saline or other fluid) and/or a gas (e.g., oxygen, air, nitrogen, etc.). Additionally or alternatively, distal end portion 140 may include one or more sensors 153 (e.g., pressure sensors, light sensors, temperature sensors, etc.). One or more lighting elements 142, one or more imaging devices 146, and distal opening 144 may have any suitable arrangement on a distal-facing surface 151 of cap portion 148. In other aspects, one or more of lighting element(s) 142, imaging device(s) 146, and/or distal opening 144 may be disposed on a radially outward-facing, or side-facing, surface 149 of cap portion 148. As will be described in further detail below, cap portion 148 may include an electrode 170 extending around at least a portion of cap portion 148 (e.g., including distal-facing surface 151 and/or side-facing surface 149 of cap portion 148). As described in further detail below, electrode 170 may be configured to cut, or cauterize, a material (e.g., tissue) using high-frequency electrical currents.

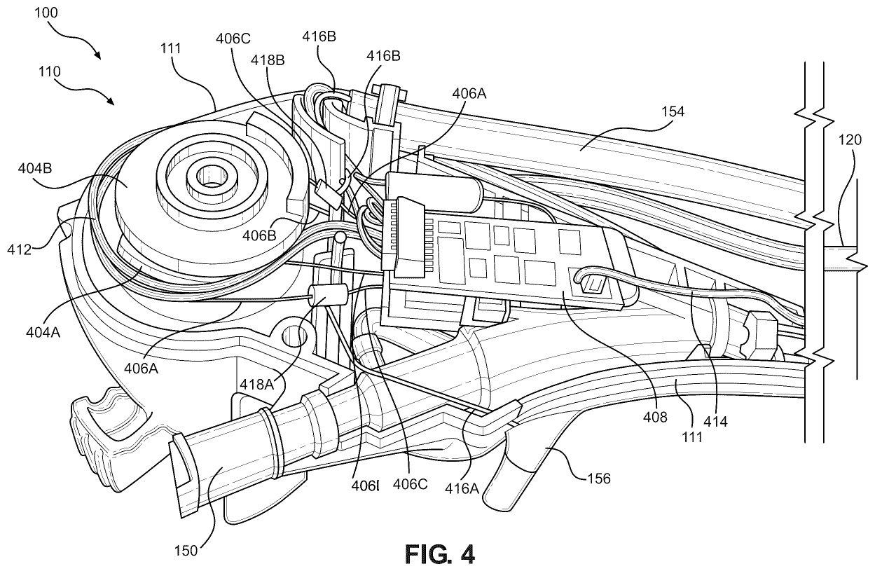

Referring to FIG. 1A, handle 110 may include one or more housings or handle shells 111 defining at least a portion of handle 110. Shown in more detail with respect to FIG. 4, the one or more handle shells 111 may contain various components related to the electrical or mechanical function of first medical device 100. Handle 110 may further include a first port, for example, a device or fluid port 150, which may provide access to a working channel of first medical device 100. In these aspects, first port 150 may be configured to deliver a fluid (e.g., a liquid or a gas), apply suction, and/or provide access to the working channel of first medical device 100 such that an accessory instrument (e.g., a basket, a guidewire, a snare, etc.) may be inserted into first port 150, advanced through the working channel of shaft 120, and out of distal opening 144 of distal end portion 140.

Handle 110 may also include one or more actuators 152 for controlling aspects of shaft 120 and/or distal end portion 140. For example, one or more actuators 152 may include one or more steering knobs (or levers, sliders, etc.) for controlling deflection, articulation, or steering of steerable section 126. Although not shown, handle 110 may include one or more locks configured to fix or secure a position or orientation of one or more actuators 152, for example, to fix or secure steerable section 126 in a desired position. For example, one or more steering cables and/or wires may extend from one or more actuators 152 to steerable section 126. Tensioning or de-tensioning of the steering cables and/or wires (e.g., via rotation or movement of the one or more actuators 152) may cause steerable section 126 to deflect in the one or more directions (e.g., up, down, left, and/or right directions). Actuator(s) 152 may additionally or alternatively control electronic or mechanical elements of distal end portion 140 or deliver air, suction, and/or water.

Handle 110 may further include a second port, for example, an electrical connector or port 156. As will be described in further detail below with respect to FIG. 4, second port 156 may be configured for coupling to the electrosurgical unit, or generator. An exemplary position of second port 156 on handle 110 is illustrated in FIG. 1A.

Second port 156 is shown as being distal to first port 150. However, second port 156 may be positioned elsewhere on handle 110. For example, second port 156 may be proximal or adjacent to first port 150. In other aspects, second port 156 may be on or extend from an alternative side of handle 110 (e.g., on a side of first medical device 100 that is facing into the page, a side of first medical device 100 that is facing out of the page, etc.).

Internal to handle 110 (shown and described in more detail with respect to FIG. 4), second port 156 may be electrically coupled to one or more metallic and/or conductive elements extending through first medical device 100. For example, second port 156 may be electrically coupled to one or more of the steering cables and/or wires configured for controlling the articulation of steerable section 126. Alternatively, second port 156 and electrode 170 may be electrically coupled to a conductive metal and/or a conductive portion or layer (e.g., a braid) of shaft 120. Accordingly, electrical current may be permitted to pass or otherwise flow from the electrosurgical unit (e.g., via second port 156), through the steering cable(s)/wire(s) or metallic and/or conductive layer of shaft 120, and to electrode 170. In these aspects, electrode 170 may be configured to cut tissue using electrical current (e.g., one or more high-frequency electrical currents). Such configurations may help to reduce, or eliminate, the need for additional wires extending through an entire longitudinal length of shaft 120.

In other aspects, second port 156 may be electrically coupled to a wire or cable extending within handle 110, longitudinally though shaft 120, and to electrode 170. Such a configuration may require the addition of one or more wires extending through shaft 120, for example, between second port 156 and electrode 170.

Referring to FIG. 1A, an umbilicus 154 may extend from handle 110. Umbilicus 154 may include one or more cables, tubes, or other conduits for conveying electrical signals, air, water, and/or suction to or from handle 110, shaft 120, and distal end portion 140. Umbilicus 154 may include one or more proximal connectors (not shown) for connecting to capital equipment, which may supply electrical controls, air, water, and/or suction.

In some configurations, second port 156 may be incorporated into umbilicus 154. For example, second port 156 may be incorporated within the proximal connector(s) of umbilicus 154 (e.g., an end of umbilicus 154 that is not connected to handle 110). Cabling associated with second port 156 may extend through a longitudinal length of umbilicus 154. In some configurations, the cabling associated with second port 156 may be electrically coupled to the steering cable(s)/wire(s), as discussed above. In other configurations the cabling associated with second port 156 may continue through shaft 120 and be directly coupled to electrode 170. In either of these configurations, for example, handle 110 may not include second port 156.

In some aspects, first medical device 100 may be used alone. For example, first medical device 100 may be inserted directly into a subject via a natural orifice or incision. As described above, one or more additional medical devices (e.g., accessory devices) may be inserted through first port 150 of first medical device 100. In other aspects, first medical device 100 may be inserted into a subject, for example, alongside (e.g., next to) a second medical device. The second medical device may be any medical device configured for insertion into a subject (e.g., a scope, a catheter, a guidewire, a basket, etc.). In other aspects, first medical device 100 may be inserted through a working channel of a second medical device.

FIG. 2 depicts a medical system 200, including first medical device 100 and a second medical device 202. As shown, second medical device 202 may be a duodenoscope. Although a duodenoscope is referenced herein, it will be appreciated that second medical device 202 may be any medical device having a working channel extending from a proximal end to a distal end, such as, for example, a ureteroscope, an endoscope, a gastroscope, an endoscopic ultrasonography (“EUS”) scope, a colonoscope, a bronchoscope, a laparoscope, an arthroscope, a cystoscope, an aspiration scope, a sheath, or a catheter.

Second medical device 202 may include a handle 210 and a shaft 220. Although not shown in FIG. 2, a distal tip having any of the features of distal end portion 140 may be at a distal end of shaft 220. Shaft 220 may be insertable into a body lumen of a subject in order to perform a procedure at a target site in the body lumen or other portion of the body.

Handle 210 may include a port 250. Port 250 may provide access to a working channel 258 of second medical device 202. A medical device (e.g., first medical device 100) may be configured to be inserted into port 250, advanced through working channel 258 of shaft 220, and extended distally from the distal tip of second medical device 202. Working channel 258 is shown in dotted lines within shaft 220 to illustrate working channel 258 extending through an interior portion of shaft 220.

Handle 210 may also include one or more actuators 252 for controlling aspects of shaft 220 and/or a distal tip of second medical device 202. For example, actuator(s) 252 may include one or more knobs, levers, sliders, joysticks, buttons, or the like for articulating a distal portion of shaft 220. One or more actuators 252 further include levers, knobs, buttons, sliders, buttons, or the like for locking the distal portion of shaft 220 from articulating. One or more actuators 252 may also include one or more actuators for capturing images or video from an imaging device of a distal tip of second medical device 202, for delivering air or water, and/or for applying suction to a distal tip of second medical device 202.

Referring still to FIG. 2, handle 110 of first medical device 100 may be coupled to handle 210 of second medical device 202. For example, a strap, clamp, or the like may be configured to couple handle 110 of first medical device 100 to handle 210 of second medical device 202. Distal end portion 140 (FIG. 1B) of first medical device 100 may be inserted into port 250 of second medical device 202. Distal portion 124 of shaft 120 of first medical device 100 may be advanced through shaft 220, for example, until distal end portion 140 extends from a distal tip of second medical device 202. Because shaft 220 may be inserted into a body lumen of a subject, distal portion 124 of shaft 120 may also be inserted into the body lumen of the subject. In examples, a total length of shaft 120 may be approximately 200 cm long, and shaft 220 may be approximately 150 cm to approximately 175 cm long. However, other lengths of each of shaft 120 and shaft 220 are contemplated. In these examples, shaft 120 may be longer than shaft 220, in order for distal end portion 140 to extend from the distal tip of second medical device 202.

As shown in FIG. 2, with distal portion 124 of first medical device 100 inserted into working channel 258 of second medical device 202, proximal portion 122 of shaft 120 of first medical device 100 may remain proximal of and outside of port 250 and outside of working channel 258 of second medical device 202. Thus, proximal portion 122 of first medical device 100 may remain external to a body (and a body lumen) of a subject. Proximal portion 122 may form a loop shape (e.g., a working loop). Handle 110 may be coupled to handle 210 distally of port 250, such that proximal portion 122 loops proximally before entering port 250. For example, proximal portion 122 may extend distally from a distal end of handle 110, and proximal portion 122 may be looped back in a proximal direction toward port 250, before then extending distally into port 250. In examples, a length of proximal portion 122 (e.g., the portion of shaft 120 that is proximal or outside of port 250 and second medical device 202) may be approximately 25 cm to approximately 40 cm, although other lengths may be contemplated.

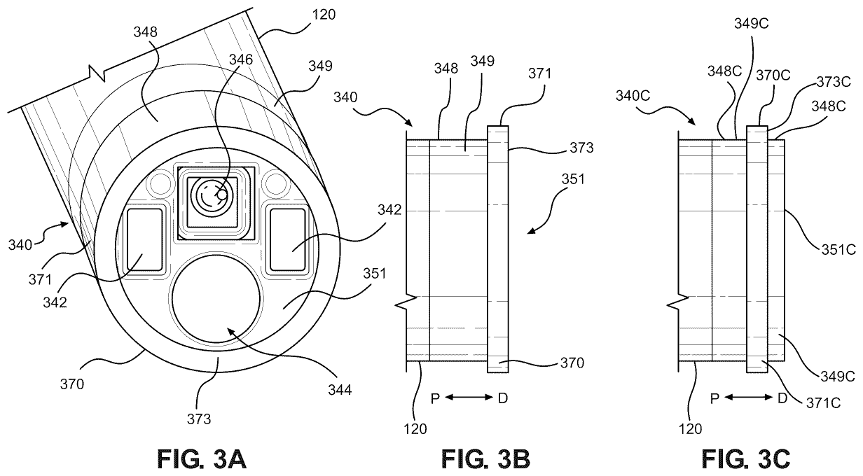

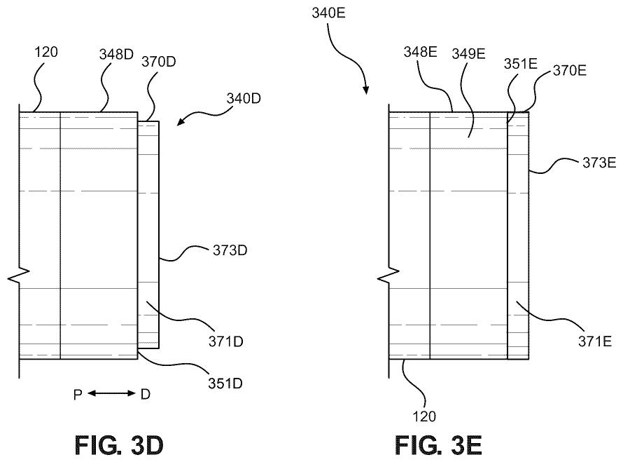

FIG. 3A illustrates a perspective view of an exemplary distal end portion 340, and FIG. 3B illustrates a side view of exemplary distal end portion 340. Distal end portion 340 may have any or all of the same characteristics as distal end portion 140 of first medical device 100, described above with respect to at least FIG. 1B. For example, distal end portion 340 may be coupled (directly or indirectly) to a distal end of shaft 120.

Shown in FIG. 3A, distal end portion 340 may include a cap 348. Cap 348 may be formed of a variety of materials having conductive or insulative properties. For example, cap 348 may be formed of a conductive metal (e.g., tungsten, silver, copper, gold, aluminum, etc.) and/or a thermoplastic material capable of being exposed to high temperatures without melting or deforming. Distal end portion 340 may include one or more lighting elements 342, one or more imaging devices 346, and a distal opening 344. An exemplary arrangement of distal end portion 340 includes two lighting elements 342, one imaging device 346, and one distal opening 344 on a distal face 351, as illustrated in FIG. 3A. However, alternative arrangements of distal end portion 340 may be contemplated. In some aspects, one or more of lighting element(s) 342, imaging device(s) 346, and/or distal opening 344 may be disposed on a radially outward-facing, or side-facing, surface 349 of cap 348.

Referring to FIGS. 3A and 3B, cap 348 may further include an electrode 370. Electrode 370 may have any or all of the same characteristics of electrode 170, discussed above with respect to FIG. 1B. For example, electrode 370 may be configured to cut tissue via electrocautery techniques. In these aspects, electrode 370 may be comprised of a metallic and/or conductive capable of receiving an electric current without deforming or melting. Such materials may include, for example, tungsten, silver, copper, gold, aluminum, etc.

Electrode 370 may extend around at least a portion of a perimeter (e.g., radial exterior) of cap 348 and/or extend distally from or relative to distal face 351. For example, electrode 370 may extend along at least a portion of side-facing surface 349 of cap 348. In some aspects, electrode 370 may be generally circular, cylindrical, or ring-shaped. In other aspects, electrode 370 may be one or more curved, or arcuate, portions extending around at least a portion of the perimeter of cap 348 (e.g., along at least a portion of side-facing surface 349 of cap 348). Electrode 370 may at least partially encompass, or surround, one or more of lighting element(s) 342, imaging device(s) 346, and/or distal opening 344. In other aspects, electrode 370 may include one or more linear or straight portions disposed along a longitudinal length of cap 348, for example, along at least a portion of side-facing surface 349.

In aspects, electrode 370 may be coupled to cap 348 (e.g., to radially outer surface 349 and/or to distal face 351). For example, electrode 370 may be welded, glued, or otherwise fixed to radially outer surface 349 and/or distal face 351. In some aspects, electrode 370 may be formed directly on to cap 348, for example, via chemical vapor deposition techniques in which a solid material is deposited using vapor produced by a chemical reaction. Furthermore, electrode 370 may be integrally formed with cap 348. For example, cap 384 may be formed of a conductive material and coated with an insulating material. A portion of cap 384 may be uncovered by or otherwise exposed from the insulating material. The uncovered portion of cap 384 may thereby form electrode 370.

In aspects, cap 348 may include a groove or a channel disposed on radially outer surface 349 and/or distal face 351. The groove or channel may be configured to receive a portion of electrode 370 (e.g., a radially inward portion of electrode 370 or a proximal portion of electrode 370). For example, a radially inward portion of electrode 370C may extend radially into cap 348, or towards a longitudinal axis of distal portion 340C.

A diameter of electrode 370 may be greater than a diameter of cap 348. For example, a radially outward surface 371 of electrode 370 may extend radially outward relative to side-facing surface 349 of cap 348. A distal surface 373 of electrode 370 may be longitudinally aligned with or distal of distal face 351 of cap 348.

FIGS. 3C-3E each illustrate a side view of various alternative configurations of a distal end portion. Reference numbers of FIGS. 3C-3E may correspond to reference numbers of distal portion 340 of FIGS. 3A and 3B, with a letter added to the end of each reference number. For example, reference numbers of FIG. 3C may include a “C” added to the end of each reference number. Additionally, reference numbers of FIG. 3D may include a “D” added to the end of each reference number, and reference numbers of FIG. 3E may include an “E” added to the end of each reference number. Corresponding reference numbers may have corresponding properties, except as explained below.

For example, referring to FIG. 3C, an exemplary distal portion 340C coupled to a distal end of shaft 120 is shown. An electrode 370C of distal portion 340C may be disposed proximally of a distalmost face 351C of a cap 348C. For example, a distal portion of cap 348C may extend distally relative to electrode 370C.

Electrode 370C may extend around at least a portion of a perimeter of cap 348C. For example, electrode 370C may extend along at least a portion of side-facing surface 349C of cap 348C. In some aspects, electrode 370C may be generally circular, cylindrical, or ring-shaped. In other aspects, electrode 370C may be one or more curved, or arcuate, portions extending around at least a portion of the perimeter of cap 348C (e.g., along at least a portion of side-facing surface 349C of cap 348C).

In aspects, electrode 370C may be coupled to cap 348C (e.g., to radially outer surface 349C). For example, electrode 370C may be welded, glued, or otherwise fixed to radially outer surface 349C. In some aspects, electrode 370C may be formed directly on to cap 348C, for example, via chemical vapor deposition techniques. In aspects, cap 348C may include a groove or a channel disposed on radially outer surface 349C. The groove or channel may be configured to receive a portion of electrode 370C (e.g., a radially inward portion of electrode 370C). For example, a radially inward portion of electrode 370C may extend radially into cap 348, or towards a longitudinal axis of distal portion 340C.

A radially outer surface 371C of electrode 370C may extend around at least a portion of an outer surface 349C of cap 348C and extend radially outward, for example, away from outer surface 349C. In these aspects, a diameter of electrode 370C may be greater than a diameter of cap 348C.

In some configurations, a diameter of an exemplary cutting portion may be less than or equal to a diameter of an exemplary cap. For example, FIG. 3D illustrates an electrode 370D having a smaller diameter as compared to a diameter of a cap 348D. A distal surface 373D of electrode 370D may be distal of a distal face 351D of cap 348D. For example, electrode 370D of FIG. 3D may extend distally of distal face 351D of cap 348D.

Electrode 370D may be generally circular, cylindrical, or ring-shaped. Accordingly, one or more lighting elements 342, one or more imaging devices 346, and a distal opening 344 of a working channel, each shown in FIG. 3A, may be disposed within a central opening of electrode 370D. In other aspects, electrode 370D may be one or more curved, or arcuate, portions extending distally from distal face 351D of cap 348D. In aspects, electrode 370D may be coupled to cap 348D (e.g., to distal face 351D). For example, electrode 370D may be welded, glued, or otherwise fixed to distal face 351D. In some aspects, electrode 370D may be formed directly on to cap 348D, for example, via chemical vapor deposition techniques. In aspects, cap 348D may include a groove or a channel disposed on distal face 351D. The groove or channel may be configured to receive at least a proximal portion of electrode 370D. For example, a proximal end of electrode 370D may extend proximally, for example, into or through cap 348D.

FIG. 3E illustrates an electrode 370E having a diameter equal to a diameter of a cap 348E. For example, a radially outer surface 371E of electrode 370E may be adjacent to or radially aligned with a radially outer surface 349E of cap 348E. A distal surface 373E of electrode 370E may be distal of a distal face 351E of cap 348E. For example, electrode 370E of FIG. 3E may extend distally from distal face 351E of cap 348E.

Electrode 370E may be coupled to cap 348E (e.g., to distal face 351D and/or to radially outer surface 349E of cap 348E. For example, electrode 370E may be welded, glued, or otherwise fixed to cap 348E. In some aspects, electrode 370E may be formed directly on to cap 348E, for example, via chemical vapor deposition techniques. In aspects, radially outer surface 349E of cap 348E may include a groove or a channel disposed on distal face 351E and/or radially outer surface 349E. The groove or channel may be configured to receive at least a proximal portion of electrode 370D. For example, a proximal end of electrode 370D may extend proximally, for example, into or through cap 348E.

Electrode 370E may be generally circular, cylindrical, or ring-shaped. Accordingly, one or more lighting elements 342, one or more imaging devices 346, and a distal opening 344 of a working channel, each shown in FIG. 3A, may be disposed within a central opening of electrode 370E. In other aspects, electrode 370E may be one or more curved, or arcuate, portions extending distally from distal face 351E of cap 348E.

Aspects of each of FIGS. 3A-3E may be combined in any configuration. For example, an exemplary distal end portion may include a single cutting portion, as illustrated in FIGS. 3A-3E. Alternatively, an exemplary distal end portion may include two or more electrodes. For example, an exemplary distal end portion may include two electrodes 370A spaced apart (e.g., radially, longitudinally, etc.) from one another. In other aspects, an exemplary distal end portion may include electrode 370A and electrode 370C or electrode 370D. Many other combinations or configurations of the distal end portion may be contemplated. The different electrodes may be configured to cut tissue using the same or different frequencies. For example, the electrodes may be configured to cut tissue at frequencies greater than approximately 50 kilohertz (kHz).

During use, any one of distal end portions 140, 340, 340C, 340D, and/or 340E of respective FIGS. 1B and 3A-3E may be navigated to a target site within a subject. Handle 110 may be used, for example, to assist in navigating distal end portions 140, 340, 340A, 340B, 340C, 340D, and/or 340E to the target site within the subject (e.g., via rotation of actuator(s) 152). Before or upon reaching the target site, distal end portions 140, 340, 340A, 340B, 340C, 340D, 340E of respective FIGS. 1B and 3A-3E may be activated to cut, or cauterize, or otherwise deliver energy to tissue, for example, endoscopically. For example, the electrosurgical unit coupled to first medical device 100 (e.g., via second port 156 and/or umbilicus 154) may deliver energy to handle 110 via umbilicus 154, and handle 110 may be electrically connected to electrode(s) via one or more conductive elements, for example, extending through shaft 120. Although not shown, the electrosurgical unit may be activated, for example, via a button and/or a foot pedal.

FIG. 4 illustrates an internal view of handle 110 of first medical device 100 of FIG. 1A. In particular, FIG. 4 illustrates an exemplary configuration of one or more cables or wires associated with second port 156 and/or umbilicus 154 (FIG. 1A) that are disposed within handle 110. As discussed with reference to FIG. 1A, handle 110 may include first port 150 and second port 156.

A first spool 404A and a second spool 404B may each be disposed within a handle shell 111 that at least partially defines handle 110. First spool 404A and second spool 404B may be independently rotatable relative to one another. One or more steering elements may at least partially surround one of first spool 404A or second spool 404B. For example, a first steering element 406A and a second steering element 406B may at least partially surround and extend distally from first spool 404A. A third steering element 406C and a fourth steering element 406D may at least partially surround and extend distally from second spool 404B.

Steering elements 406A, 406B, 406C, 406D may extend from spools 404A, 404B, respectively, and towards shaft 120. For example, steering elements 404 may extend distally through one or more lumens of shaft 120 and to distal end portion 140 (FIG. 1B). In these aspects, rotation of first spool 404A and/or second spool 404B may translate or otherwise urge one or more steering elements 404 proximally and/or distally. Accordingly, distal end portion 140 (FIG. 1B) may be articulated, or bent, in one or more directions (e.g., up, down, left, and/or right).

A circuit board 408 may be disposed within handle 110. In some aspects, a cable 412 may extend from umbilicus 154, around spools 404A, 404B (i.e., within a proximal portion of handle 110), and to circuit board 408. One or more wires 414 may extend distally from circuit board 408, for example, through one or more lumens of shaft 120. Circuit board 408 may include circuitry, for example, to permit the use of imaging device(s) 146 and/or lighting element(s) 142 of distal end portion 140 (FIG. 1B).

Referring still to FIG. 4, second port 156 may be electrically coupled (directly or indirectly) to one or more steering elements configured for controlling the articulation of steerable section 126 (FIG. 1B). For example, a first connecting element 416A may extend between second port 156 and first steering element 406A. A first coupler 418A may be fixed to first steering element 406A. First coupler 418A may be disposed within a space defined within handle 110, for example, between circuit board 408 and spools 404A, 404B.

Although not shown, electrode 170 (FIG. 1B) (or any other cutting portion described herein) may be electrically connected to a distal end of first steering element 406A. For example, first steering element 406A may be at least partially conductive and may extend through a lumen of shaft 120. In some aspects and as discussed below, first steering element 406A (or one or more other steering elements) may include a conductive inner portion and an insulative outer portion. First steering element 406A may terminate proximally of, or within, cap portion 148 of distal end portion 140 (FIG. 1B). Additional cabling or conductive element(s) within distal end portion 140 may extend between the distal end of first steering element 406A and electrode 170 (i.e., at least partially within cap portion 148). Accordingly, electrode 170 may be electrically connected to second port 156 via first steering element 406A.

Additional configurations of handle 110 are also contemplated. For example, first coupler 418A may be alternatively fixed or otherwise electrically connected to one or more of second steering element 406B, third steering element 406C, or fourth steering element 406D, which may in turn be electrically connected to electrode 170 (e.g., either directly or indirectly).

In an alternative configuration, first connecting element 416A may extend between second port 156 and a metallic and/or conductive layer, or portion, of shaft 120. In such a configuration, electrode 170 (of distal end portion 140) may be electrical coupled to the metallic and/or conductive layer of shaft 120 at a distal end of shaft 120. For example, additional cabling or conductive element(s) may extend between the metallic and/or conductive layer of shaft 120 and electrode 170 within distal end portion 140 (i.e., within cap portion 148).

In a further alternative configuration, first connecting element 416A may extend between second port 156 and may be fixed directly to electrode 170 (FIG. 1B), for example, within distal end portion 140. For example, first connecting element 416A may extend from second port 156, through a lumen of shaft 120, and be fixed directly to electrode 170.

An additional or alternative configuration is also illustrated in FIG. 4. For example, with second port 156 incorporated into umbilicus 154 (as discussed above), a second connecting element 416B may extend from a lumen of umbilicus 154 to a second coupler 418B. Second coupler 418B may be fixed to third steering element 406C (as shown) or to any other steering element that extends between handle 110 and electrode 170 of distal end portion 140 (of FIG. 1B).

In an alternative configuration, second connecting element 416B may be electrically coupled to electrode 170, for example, via the metallic and/or conductive portion or layer of shaft 120. In a further alternative configuration, second connecting element 416B may extend through shaft 120, such that second connecting element 416B is coupled to electrode 170 within distal end portion 140.

In some aspects, two or more configurations discussed herein may be used in conjunction, for example, in a configuration of distal end portion 140 that includes multiple electrodes 170. For example, a first electrode 170 may be electrically coupled to first coupler 418A, and a second electrode 170 may be electrically coupled to second coupler 418B. Accordingly, two different frequencies, powers, etc. of energy may be used simultaneously or separately to cut, cauterize, or otherwise deliver energy to tissue.

First medical device 100 may be subjected to the International Electrotechnical Commision (IEC) 60601 standard (or to other similar standards). Such a standard may require a dielectric withstand test, for example, to ensure first medical device 100 is safe for medical use. In these aspects, any of the metallic and/or conductive elements (e.g., wires or cables) directly or indirectly electrically coupled to electrode 170 of first medical device 100 may be coated with an enamel or other insulating material. For example, steering elements 406A, 406B, 406C, and 406D, connecting elements 416A, 416B, couplers 418A, 418B, the metallic and/or conductive portion or layer of shaft 120 discussed herein, and/or any additional electrical wires coupled (directly or indirectly) to electrode 170 may be coated with an enamel. The enamel may include polyurethane wire enamels, modified polyester enamels, polyesterimide enamels, polyamide-imide enamels, and/or any other enamel that has a dielectric withstand rating of approximately 4 kV/mm.

In aspects, the enamel of couplers 418A, 418B, connecting elements 416A, 416B, steering elements 406A, 406B, 406C, 406D, the metallic and/or conductive portion or layer of shaft 120, and/or any additional electrical elements fixed to electrode 170 may add approximately 3-5 kV of withstand voltage to first medical device 100. The enamel may prevent high-frequency electrical currents (e.g., from the electrosurgical unit or other devices electrically coupled to first medical device 100) from travelling to unintended components within first medical device 100 and/or to the user or subject.

In some aspects, one or more surfaces of the electrode(s) may be coated with or include the enamel. For example, a distal-facing surface (e.g., distal surface 373 of electrode 370 shown in FIGS. 3A and 3B) and/or a proximal-facing surface of the electrode (e.g., a surface that is on a side of electrode 370 opposite of distal surface 373) may be coated with the enamel. In these aspects, only tissue contacting a radially outward-facing surface of the electrode (e.g., radially outward facing surface 371 of electrode 370) may be cut, cauterized, or otherwise treated with energy.

Couplers 418A, 418B, connecting elements 416A, 416B, steering elements 406A, 406B, 406C, 406D, the metallic and/or conductive portion or layer of shaft 120, and/or any additional wires fixed to electrode 170 may, additionally or alternatively, include a coating, a sheath, and/or additional layers of material(s). In these aspects, the coating, sheath, and/or additional layers of material may help, for example, to ensure first medical device 100 is safe for use and/or to comply with IEC 60601 standard requirements.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
a handle; and
an insertion portion extending from the handle, wherein the insertion portion includes:
a shaft having a proximal end and a distal end, wherein a distal portion of the shaft includes an articulation portion, wherein the articulation portion is configured to bend the distal portion of the shaft in at least two directions; and
a cap fixed to the distal end of the shaft, wherein the cap includes: (i) an imaging device; (ii) a lighting element; and (iii) an electrode, wherein the electrode is configured to cut a material using a high-frequency electrical current.

2. The medical device of claim 1, wherein the imaging device, the lighting element, and the electrode are disposed on a distalmost surface of the cap.

3. The medical device of claim 1, wherein the electrode is ring-shaped.

4. The medical device of claim 3, wherein the electrode at least partially encompasses both the imaging device and the lighting element.

5. The medical device of claim 3, wherein the electrode extends distally from a distalmost surface of the cap.

6. The medical device of claim 5, wherein a diameter of the electrode is less than a diameter of the cap.

7. The medical device of claim 1, wherein the electrode extends around at least a portion of a radially outward-facing surface of the cap.

8. The medical device of claim 7, wherein a radially outward-facing surface of the electrode is radially aligned with a radially outward-facing surface of the cap.

9. The medical device of claim 1, wherein a distal face of the electrode is proximal of a distalmost surface of the cap.

10. The medical device of claim 1, wherein a diameter of the electrode is greater than a diameter of the cap.

11. The medical device of claim 1, wherein at least a portion of the electrode is coated with an insulating material.

12. The medical device of claim 1, wherein the medical device is a first medical device, and wherein the insertion portion is configured for insertion through a working channel of a second medical device.

13. The medical device of claim 1, wherein the handle includes an electrical port electrically coupled to the electrode.

14. The medical device of claim 13, wherein the electrical port is electrically coupled to the electrode via a steering element extending distally from the handle.

15. The medical device of claim 1, wherein the electrode is disposed longitudinally along a radially outward-facing surface of the cap.

16. A distal end portion of a medical device, comprising:
an articulation portion; and
a cap portion fixed to a distal end of the articulation portion, wherein the cap portion includes:
an imaging device;
a lighting element; and
an electrode, wherein the electrode is configured to cut tissue using a high-frequency electrical current, and
wherein the electrode is disposed on a radially outward-facing surface of the cap portion.

17. The distal end portion of claim 16, wherein the imaging device and the lighting element are disposed on a distalmost surface of the cap portion.

18. The distal end portion of claim 17, wherein a diameter of the electrode is greater than a diameter of the cap portion.

19. A medical system, comprising:
a first medical device, wherein the first medical device includes:
a first handle; and
a first shaft extending from the first handle and having a working channel; and
a second medical device, wherein the second medical device includes:
a second handle; and
a second shaft extending from the second handle, wherein the second shaft is configured for insertion through the working channel of the first medical device, wherein a distal end portion of the second shaft includes a cap, wherein the cap includes: (i) an imaging device; (ii) a lighting element; and (iii) an electrode, wherein the electrode is configured to cut a material using high-frequency electrical currents.

20. The medical system of claim 19, wherein the electrode is positioned proximal of the imaging device and the lighting element.
